(19) United States
(12) Patent Application Publication
(10) Pub. No.: US 2008/0319366 A1
(43) Pub. Date: Dec. 25, 2008

(54) ROMPER FOR USE OVER SPICA CAST

(76) Inventors: Nicole Kujawa, Waukesha, WI (US); Barbara Merriam, Oshkosh, WI (US)

Correspondence Address: BOYLE FREDRICKSON S.C., 840 North Plankinton Avenue, MILWAUKEE, WI, 53203, US

(21) Appl. No.: 12/143,925

(22) Filed: Jun. 23, 2008

Related U.S. Application Data
(60) Provisional application No. 60/945,427, filed on Jun. 21, 2007.

(51) Int. Cl. A61F 5/00; A41D 11/00

(52) U.S. Cl. 602/24; 2/80

(57) ABSTRACT

The present invention overcomes the lack of appropriate clothing for children being treated for hip dysplasia with a spica cast, Pavlic harness, or the like. A romper is disclosed that is sized to fit a child and sized to fit over a corrective device used to treat hip dysplasia. The romper of the present invention provides the convenience and neat appearance of infant clothing that is currently not available for children wearing a spica cast, Pavlic harness, or the like.

### CROSS-REFERENCE TO RELATED APPLICATION

This application claims priority under 35 U.S.C. §119(e) to U.S. Provisional Application Ser. No. 60/945,427, filed Jun. 21, 2007 and entitled “ROMPER FOR USE OVER SPICA CAST,” the contents of which are hereby incorporated by reference in their entirety.

### BACKGROUND OF THE INVENTION

1. Field of the Invention

The present invention relates to apparel, and more particularly, relates to a romper for wear over a spica cast.

2. Background

Development dislocation of the hip, or hip dysplasia, is a congenital disorder that affects about 1 out of every 1000 infants. Treatment for the disorder can include use of a Pavlik harness at birth and, if ineffective to resolve the problem, use of a spica cast between the age of 1 month up to 2 years of age. Both a Pavlik harness and spica cast keep the hips in a flexed and abducted state in an effort to maintain the ball-shaped head of the femur in the acetabulum, the socket of the pelvis.

3. Discussion of the Related Art

Children who undergo treatment with a Pavlik harness and/or spica cast are difficult to diaper, bathe, and clothe. In the past, to fit clothing over a Pavlik harness, it has been necessary to purchase clothing, generally with an elastic waist, in a size several times larger than the child. These clothes suffer from the drawbacks of being too long in the leg for the affected child, thus giving a sloppy appearance, and failing to offer the amenities present in infant clothing, making diapering the child a more difficult task.

For children being treated with a spica cast, even clothes several times too large do not fit over the cast due to the shape of the cast. As a result, children fitted with a spica cast can not wear pants of any type on their lower portion, and have been required to wear oversized tee-shirts and dresses, which, as noted above, are sized incorrectly for children, yielding overly long arms and gaping necklines. In addition to appearing undressed or sloppy, practical problems emerge from a lack of covering over the cast. Specifically, it is difficult to secure a diaper in place over the diaper hole of the cast. Food spills, drool, spit-up, and other liquids may drip onto the cast and further degrade its appearance. In addition, these liquids can drip into the cast at the upper opening and cause irritation for the child. Toys and other foreign objects can likewise fall into the cast.

What is needed is a garment that will fit over a spica cast yet fit the child within the cast to protect the cast from stains and foreign objects, help keep the child's diaper in place, provide easy access to the diaper, and give the child a “dressed” and tailored appearance.

### SUMMARY OF THE INVENTION

The present invention overcomes the lack of appropriate clothing for children being treated for hip dysplasia by providing a romper that is sized to fit the child, fit over the corrective device, be it harness or cast, and incorporate the features of infant clothing.

### BRIEF DESCRIPTION OF THE DRAWINGS

Preferred exemplary embodiments of the invention are illustrated in the accompanying drawings, in which like reference numerals represent like parts throughout, and in which:

FIG. 1 is a perspective view of a child wearing an embodiment of the romper of the present invention. The treatment device being covered by the romper is shown in dashed lines.

FIG. 2 is a perspective view of a child in a spica cast showing the details of the cast.

FIG. 3 is a front view of an embodiment of the romper, again showing the treatment device being covered by the romper in dashed lines.

FIG. 4 is a front view of a spica cast with one embodiment of the romper shown in dashed lines.

FIG. 5 is a front view of a pattern of one embodiment of the romper.

FIG. 6 is a cross sectional view taken from lies 6-6 in FIG. 3 showing the mating connection between the snap closures present in one embodiment of the invention.

FIG. 7 is the pattern shown in FIG. 5 with additional notations regarding preferred dimensions of an embodiment of the invention.

### DETAILED DESCRIPTION OF THE PREFERRED EMBODIMENTS

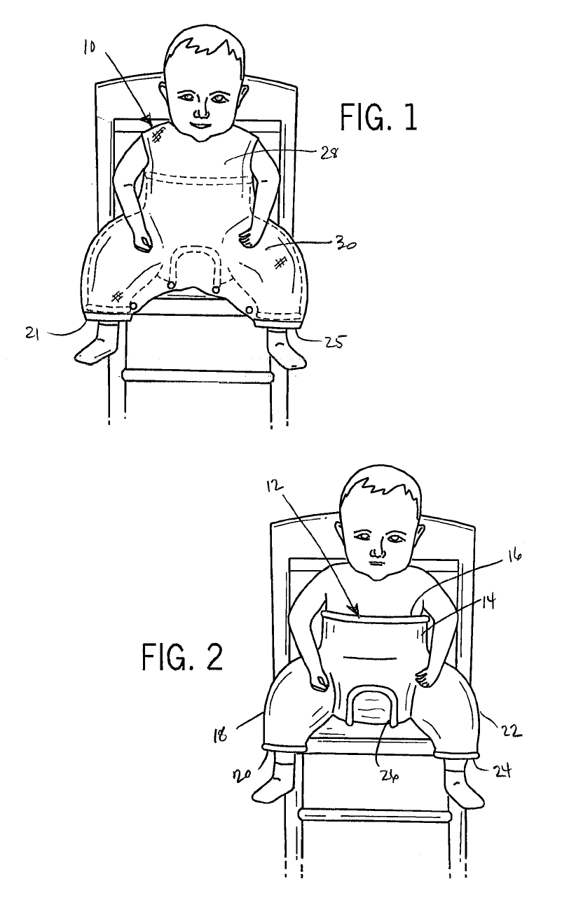

As seen in FIG. 1, the romper 10 of the present invention covers a spica or body cast 12, shown in FIG. 2. Romper 10 may also be used in connection with covering a Pavlik harness or other treatment device, but is shown in use with a cast 12 and will therefore be discussed accordingly.

Cast 12 has a waist portion 14 and a torso aperture 16, a left leg portion 18 and an aperture for the left foot 20, a right leg portion 22 and an aperture for the right foot 24, and a diaper opening 26. Romper 10 has an upper portion 28 that generally covers the patient above torso aperture 16 and a neck opening 32. Romper 10 also has a lower portion 30 that generally covers the patient from torso aperture 16 to foot apertures 20, 24, including foot openings 21, 25.

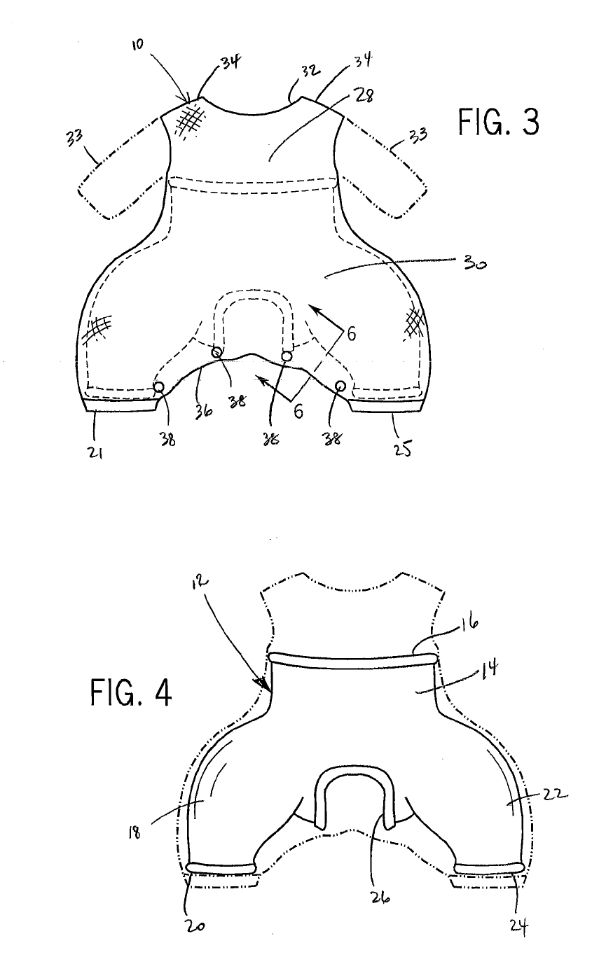

Upper portion 28 may have a neck opening 32 with a ribbed trim for comfort and stretch, or may be seamed flat, or have any other known opening shape or trim, including a front or back placket incorporating buttons or snaps, a collar or piping, or other useful or ornamental elements. Romper 10 may be fitted with sleeves 33 of any design and length, as shown in phantom in FIG. 3, or may be left sleeveless. Upper portion 28 has shoulder seams 34 that can also be constructed to include snaps or other closures at either or both shoulders for fitting romper 10 over a child's head. Upper portion 28 may be unitary with lower portion 30 or constructed of the same material as lower portion 30, but could also be constructed of a different material and either permanently or removably attached to lower portion 30. Upper portion 28 may have any number of decorative elements incorporated therein, such as straps or tie-closures at the shoulders to imitate bib overalls or a sundress, smocking or embroidery, or any other suitable useful or ornamental elements known in infant/toddler clothing design.

Lower portion 30 of romper 10 has an inseam 36 between foot openings 21, 25 that is preferably selectively openable such as is common in infant clothing for making access to a child's diaper easier. Seam 36 is selectively openable by way or one or more closures 38 along seam 36, such as snaps, as shown in the drawings and as is typical in infant clothing. Of course, closures 38 may consist of any type of suitable closure, such as hook-and-loop tape (Velcro® tape), buttons, zippers, or the like, and may be provided in any number or arrangement suitable to open and close seam 36 with relative ease.

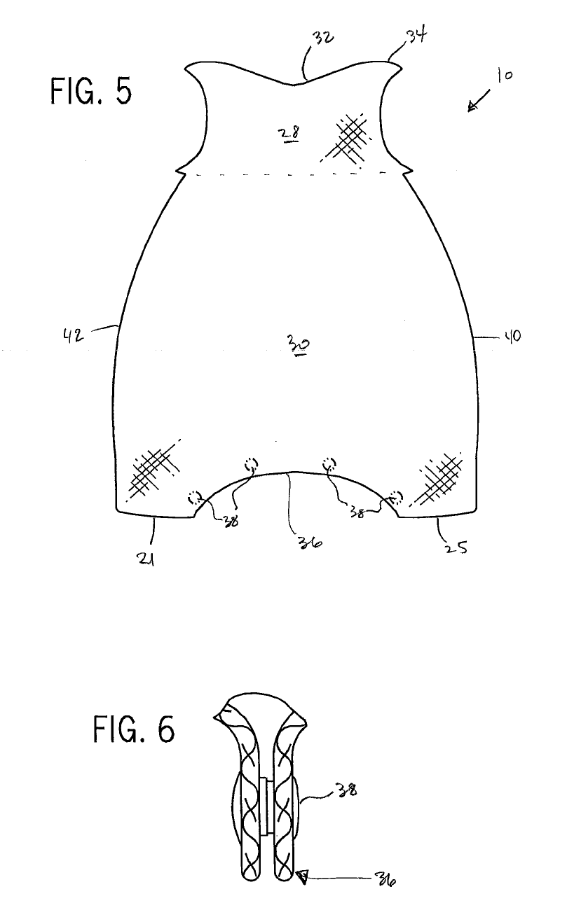

Turning now to FIG. 5, one side of a pattern for constructing romper 10 is shown. In its simplest form, romper 10 is constructed of two identical pieces of material such that the pattern shown in FIG. 5 is the same for both the front and the back of romper 10 and each piece of material is attached to the other by way of a left side seam 40 and a right side seam 42 in addition to the previously discussed shoulder seams 34 and inseam 36. Alternately, it is possible to construct romper 10 with a single piece of material having only vertical seam, or in any other suitable way known in the arts of sewing and constructing infant/toddler clothes.

Because treatment with cast 12 may last for up to two years, it is often necessary to re-cast a patient due to growth as well as for sanitary purposes. Romper 10 is therefore available in a variety of sizes, preferably from newborn to size 2T, to accommodate children during the entire treatment period. However, although some size variation is therefore contemplated due to sizing of the garment to the child, the ratio of the size of upper portion 28 to lower portion 30 is approximately 1.5 across all size ranges. This is a significantly different ratio than is typically found in infant and toddler clothing—although clothing for small children is usually somewhat wider at the bottom to accommodate bulky diapers, no more than a 1.2 ratio is typical.

For example, in the pattern shown at FIG. 5, which is approximately to scale, romper 10 is 8.4 cm across at the widest part of lower portion 30, while the widest part of upper portion 28 is 5.4 cm, providing a 1.55 ratio of lower to upper. In practice, a 2T size romper of the present invention is approximately 19.5 inches across at the widest part of lower portion 30, and 13 inches across at the widest part of upper portion 28, a ratio of 1.5 lower to upper. A ratio ranging from 1.3 to 1.6 is typically contemplated, however, it may be determined that, due to the hand-casting of patients, enough variation in the thickness and resulting width of the cast exists that a ratio of above 1.6 is required to accommodate the largest of casts while still providing a “normal” fit for the child at the upper portion 28.

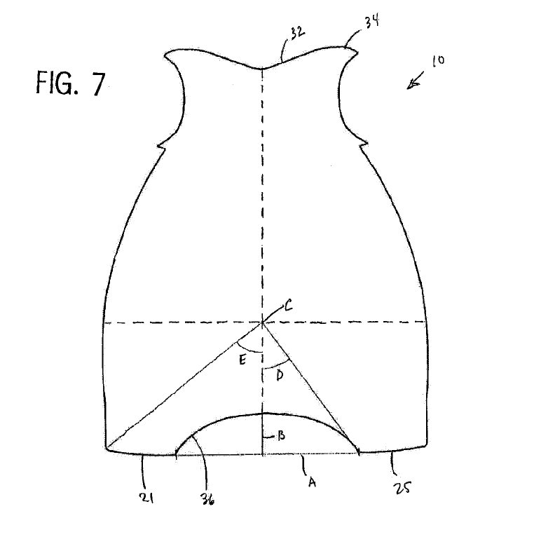

Turning now to FIG. 7, further preferred dimensions are shown. The length A between leg openings 21, 25 is, for example, approximately 11 inches, and the height B of the opening between the legs is, at its deepest point, approximately 3 inches. Point C represents the intersection of the midline of romper 10 and the line at which lower portion 30 no longer curves at side seams 40, 42. Angle D is measured from the midline and the intersection of a line between point C and the innermost portion of leg openings 21, 25. In a preferred embodiment, angle D is typically approximately 35°. Angle E is measured from the midline and the intersection of a line between point C and the outermost portion of leg openings 21, 25. In a preferred embodiment, angle E is typically approximately 50°.

It should be understood that the invention is not limited to the details of construction and arrangements of the components as set forth herein. The embodiments described herein simply explain the best modes known for practicing the invention and will enable others skilled in the art to utilize the invention. The invention is capable of other embodiments and of being practiced or carried out in various ways, and all such variations and modifications should be considered within the scope of the present invention as set forth by the claims below.

We claim:

1. A clothing garment for use with a child fitted with a medical device that sets the lower extremities of the child at a flared position, comprising:
an upper portion proportionally sized for a torso region of the child; and
a lower portion extending from the upper portion and including a pair of leg sections spaced from one another in a flared manner to accommodate the flared position of the lower extremities of the child when the child is wearing the medical device that fixes the lower extremities of the child in the flared position.

2. The garment of claim 1 wherein the lower portion is split into a first half and a second half separated from one another by a closable seam.

3. The garment of claim 2 further comprising a series of snaps fit along the closable seam to selectively join and separate the first and the second halves.

4. The garment of claim 1 wherein the upper portion has a first width and the lower portion has a second width that is approximately 1.3 to 1.8 times wider than the first width.

5. The garment of claim 4 wherein the second width is approximately 1.5 times wider than the first width.

6. A garment for wear over a body cast covering a portion of the body comprising:
a first portion designed to fit over a non-casted portion of a body;
a second portion designed to fit over a casted portion of the body, wherein the second portion is at least 1.5 times the width of the first portion.
